(19) United States
(12) Patent Application Publication
(10) Pub. No.: US 2021/0169957 A1
(43) Pub. Date: Jun. 10, 2021

(54) COMBINED USE OF SQUIRREL POXVIRUS AND MYXOMA POXVIRUS, FOR TREATING CANCER

(71) Applicant: VIROCURE, INC., Seoul (KR)

(72) Inventor: Manbok KIM (deceased), Seoul (KR)

(73) Assignee: VIROCURE, INC., Seoul (KR)

(21) Appl. No.: 17/250,667

(22) PCT Filed: Jul. 30, 2019
(86) PCT No.: PCT/KR2019/009459

§ 371 Date: Feb. 17, 2021

Related U.S. Application Data
(60) Provisional application No. 62/719,342, filed on Aug. 17, 2018.

(51) Int. Cl. A61K 35/768; A61K 9/00; A61P 35/00

(52) U.S. Cl. CPC A61K 35/768; A61P 35/00; A61K 9/0019

(57) ABSTRACT

Provided is a composition and method for treating cancer in a patient in need of cancer treatment, and the cancer can be treated by a combined therapeutic use of squirrel poxvirus and myxoma poxvirus.

The present invention relates to a therapeutic use of squirrel poxvirus and myxoma poxvirus for cancer treatment.

This application claims priority to and the benefit of U.S. Patent Application No. 62/719,342, filed on Aug. 17, 2018, the disclosure of which is incorporated herein by reference in its entirety.

### BACKGROUND ART

### Technical Field

Cancer is generally defined as a disease in which cells divide at an excessive rate and their functions become abnormal, malignant tumors and neoplasms. Standard therapeutic methods include surgery, chemotherapy and radiation therapy that remove affected cancerous tissue. Specific types of cancer do not response to such chemotherapy, radiation therapy and other treatments or have resistance thereto. Although some human tumors are sensitive to conventional chemo/radiation therapies, it is known that various solid tumors (e.g., a brain tumor, a breast tumor, an ovarian tumor and other tumors) and blood tumors are not treated by conventional treatment regimens. Leukemia is an exemplary cancer of the blood or bone marrow, characterized by abnormal proliferation (production by proliferation) of blood cells, commonly, white blood cells. Chronic myeloid leukemia (CML) is a type of leukemia characterized by a considerable increase in bone marrow cells and uncontrollable growth of bone marrow cells in the bone marrow, and accumulation of the cells in blood. In addition, multiple myeloma (MM) is incurable cancer and a clonal B-cell neoplasm affecting finally differentiated B cells (that is, plasma cells). In the multiple myeloma, clusters of excessive cancerous plasma cells may form tumors in the bone marrow, called myeloma. Therefore, the presence of many tumors is called multiple myeloma. The average survival rate for patients with multiple myeloma is approximately 3 years. Genetic studies elucidated that multiple myeloma tumorigenesis occurs due to step-wise malignant transformation including oncogene overactivation and inactivation of various tumor suppressor genes. Despite a variety of medications, cancer patients (CML or MM) often acquire drug resistance quickly, and suffer from serious side effects caused by long-term drug treatment.

Naturally-occurring viruses are live, replication-proficient viruses that specifically infect human cancer cells and avoid their normal counterparts. Since the discovery of a natural oncotropic viruses in 1920, various replicating viruses have shown various degrees of safety and efficacy in preclinical or clinical applications for human or animal anticancer therapies. Cellular oncogenes such as Ras and c-Myc are known as host genes playing an important role in determining the tropism of oncolytic viruses. Significantly, it was discovered that cellular tumor suppressor genes also play an important role in determining the tropism of oncolytic viruses (Kim M et al., Oncogene. 29:3990-3996, 2010). Carcinogenesis is a multi-step process involving abnormal accumulation of cancer genes and abnormal accumulation of tumor suppressor genes. Therefore, it is interesting that oncolytic viruses are known to utilize signaling between an abnormal oncogene and a cell tumor suppressor, which is not frequently regulated in gynecologic malignancies in terms of determination of oncolytic specificity and efficacy of the viruses. Unlike oncogenes, many tumor suppressor genes such as p53, ATM and RB are known to play an important role in genome fidelity/maintenance (Kim M et al., Oncogene. 29:3990-3996, 2010). Accordingly, tumor suppressor gene abnormalities may affect host genomic integrity, and similarly, destroy an intact antiviral network due to the accumulation of genetic defects causing natural viral anti-tumor effects.

Since the eradication of smallpox in the 1970s, the oncolytic property of replicating vaccinia viruses and other poxviruses have been confirmed. According to the molecular research for the past two decades, currently, the development of cellular oncogenes and tumor suppressor genes are quite well established in order to determine of the oncolytic tropism of poxviruses. Today, in North America, Europe and a few Asian countries, clinical trials are being conducted using modified versions of vaccina virus strains. Although vaccina virus complications could take place in immunocompromised individuals on a large population scale, the safety aspects of the attenuated version of rare vaccinia viruses are now well established through many experiments that have been conducted previously (Jacobs, B. L. et al, Antiviral Res. 84, 1-13, 2009; Nalca, A., Zumbrun, E. E., Drug Des Devel Ther. 4:71-79, 2010).

### DISCLOSURE

### Technical Problem

The inventors confirmed that the combination of squirrel poxvirus and myxoma poxvirus exhibited a remarkable synergistic effect for treating cancer, and based on this, the present invention was completed.

The present invention is directed to providing a pharmaceutical composition for preventing or treating cancer, which comprises a therapeutically effective amount of (a) a combination of squirrel poxvirus and myxoma poxvirus or (b) a biological sample treated with (a) as an active ingredient.

The present invention is also directed to providing a method of treating cancer of a patient, which comprises administering a therapeutically effective amount of (a) a combination of squirrel poxvirus and myxoma poxvirus or (b) a biological sample treated with (a) to a patient in need thereof.

The present invention is also directed to providing a use for preventing or treating cancer of composition comprising a therapeutically effective amount of (a) a combination of squirrel poxvirus and myxoma poxvirus or (b) a biological sample treated with (a) as an active ingredient.

The present invention is also directed to providing a kit which comprises the pharmaceutical composition described above and an indicating label for the composition.

However, technical problems to be solved in the present invention are not limited to the above-described problems, and other problems which are not described herein will be fully understood by those of ordinary skill in the art from the following descriptions.

### Technical Solution

To attain the above-described objects, one aspect of the present invention provides a method of treating cancer of a patient, which comprises administering a therapeutically effective amount of (a) a combination of squirrel poxvirus and myxoma poxvirus or (b) a biological sample treated with (a) to a patient in need thereof.

Another aspect of the present invention provides a use for preventing or treating cancer of composition comprising a therapeutically effective amount of (a) a combination of squirrel poxvirus and myxoma poxvirus or (b) a biological sample treated with (a) as an active ingredient.

Still another aspect of the present invention provides a pharmaceutical composition for preventing or treating cancer, which comprises a therapeutically effective amount of (a) a combination of squirrel poxvirus and myxoma poxvirus or (b) a biological sample treated with (a), and a pharmaceutically acceptable excipient. In the pharmaceutical composition, a pharmaceutically acceptable excipient may be comprised. In some embodiment, the pharmaceutical composition is an injectable form. In some embodiments, the pharmaceutical composition is an oral form. In addition, in the present invention, a kit including the above-described pharmaceutical composition and an indicating label for the composition is disclosed.

In some embodiments, squirrel poxvirus comprises wild-type squirrel poxvirus. In some embodiments, the squirrel poxvirus comprises a therapeutic gene-expressing squirrel poxvirus for targeting cancer. In some embodiments, myxoma poxvirus comprises wild-type myxoma poxvirus. In some embodiments, myxoma poxvirus comprises a therapeutic gene-expressing myxoma poxvirus for targeting cancer. In some embodiments, the squirrel poxvirus may be contained at 3×10⁵ to 10×10⁵ TCID₅₀. In some embodiments, the myxoma poxvirus may be contained at 0.1×10⁵ to 10×10⁵ TCID₅₀. In some embodiments, a potency ratio of the squirrel poxvirus to the myxoma poxvirus in the combination of the squirrel poxvirus and the myxoma poxvirus may be 1:1 to 100:1 (squirrel poxvirus:myxoma poxvirus).

In some embodiments, cancer may be tumor suppressor-deficient cancer. In some embodiments, cancer may be selected from hematopoietic malignancies, lung cancer, liver cancer, breast cancer, colon cancer, pancreatic cancer, brain cancer, gynecological cancer or stomach cancer. In some embodiments, the cancer may be a hematopoietic malignancy selected from the group consisting of myeloproliferative disorders such as lymphoma, myeloma, myelodysplastic syndrome (MDS) and polycythemia vera, and leukemia. Cancer cells may be malignant or benign cells. In some embodiments, the cancer may be an incurable cancer comprising oncolytic virus-resistant cancer.

In some embodiments, the biological sample is prepared to kill a plurality of cancer cells by applying an effective amount of the combination of squirrel poxvirus and myxoma poxvirus to an ex vivo biological sample.

### Advantageous Effects

The combination of squirrel poxvirus and myxoma poxvirus according to the present invention exhibit a significant synergistic effect in cancer treatment.

### DESCRIPTION OF DRAWINGS

FIG. 1 shows a squirrel poxvirus oncolytic effect in gynecological carcinoma (EM magnification: 30,000×).

FIG. 2 shows a squirrel poxvirus oncolytic effect in hepatocellular carcinoma (EM magnification: 60,000×).

FIG. 3 shows a squirrel poxvirus oncolytic effect in stomach cancer (EM magnification: 30,000×).

FIG. 4 shows a synergistic oncolytic effect of squirrel poxvirus and myxoma poxvirus in Hep3B hepatocellular carcinoma (SQPV: squirrel poxvirus; MYXVgfp: gfp-expressing myxoma poxvirus).

FIG. 5 shows a synergistic oncolytic effect of squirrel poxvirus and myxoma poxvirus in A549 lung cancer (SQPV: squirrel poxvirus; MYXVgfp: gfp-expressing myxoma poxvirus).

FIG. 6 shows a synergistic oncolytic effect of squirrel poxvirus and myxoma poxvirus in MCF7 breast cancer cells (SQPV: squirrel poxvirus; MYXVgfp: gfp-expressing myxoma poxvirus).

FIG. 7 shows a synergistic oncolytic effect of squirrel poxvirus or myxoma poxvirus in squirrel poxvirus-resistant cancer (SQPV: wild-type squirrel poxvirus; MYXVgfp: gfp-expressing myxoma poxvirus).

FIGS. 8A and 8B show a synergistic oncolytic effect of squirrel poxvirus and myxoma poxvirus in in vivo xenograft mouse models (vehicle: negative control; SQPV: squirrel poxvirus; MYXVgfp: gfp-expressing myxoma poxvirus).

### MODES OF THE INVENTION

Surprisingly, it was found that the combined use of squirrel poxvirus and myxoma poxvirus remarkably improves oncolytic potential against human cancer including an oncolytic virus-resistant tumor, which reveals that the combination of squirrel poxvirus and myxoma poxvirus may exhibit significantly improved oncolytic potential against various types of human or animal cancer. Moreover, non-vaccinia poxviruses, for example, squirrel poxvirus and myxoma poxvirus, are non-pathogenic to a human, and have oncolytic potential against various types of cancer, and thus are expected to be effective.

The present invention provides a method of treating cancer of a patient, which comprises: administering a therapeutically effective amount of (a) a combination of squirrel poxvirus and myxoma poxvirus or (b) a biological sample treated with (a) to a patient in need thereof.

In addition, the present invention provides a use for preventing or treating cancer of composition comprising a therapeutically effective amount of (a) a combination of squirrel poxvirus and myxoma poxvirus or (b) a biological sample treated with (a) as an active ingredient.

The present invention also provides a pharmaceutical composition for preventing or treating cancer, which comprises a therapeutically effective amount of (a) a combination of squirrel poxvirus and myxoma poxvirus or (b) a biological sample treated with (a), and a pharmaceutically acceptable excipient. In the pharmaceutical composition, a pharmaceutically acceptable excipient may be comprised. In some embodiment, the pharmaceutical composition is an injectable form. In some embodiments, the pharmaceutical composition is an oral form. In addition, in the present invention, a kit comprising the above-described pharmaceutical composition and an indicating label for the composition is disclosed.

Cancer may be cancer having a tumor suppressor-deficient function. The cancer may be selected from hematopoietic malignancies, lung cancer, liver cancer, breast cancer, colon cancer, pancreatic cancer, brain cancer, gynecological cancer or stomach cancer. The cancer may be a hematopoietic malignancy selected from the group consisting of myeloproliferative disorders such as lymphoma, myeloma, myelodysplastic syndrome (MDS) and polycythemia vera, and leukemia. Cancer cells may be malignant or benign cells. In some embodiments, the cancer may be an incurable cancer including oncolytic virus-resistant cancer.

The term “squirrel poxvirus” refers to a squirrel poxvirus strain, which is not pathogenic to a human or a non-squirrel animal. Specific animal poxviruses affect only specific animal species (Kim M., 2015). In other words, the viruses do not cause diseases in a human or non-tropismic animal in nature. In some embodiments, squirrel poxvirus includes at least one of a wild-type squirrel poxvirus strain and a manipulated squirrel poxvirus strain containing a therapeutic gene for targeting cancer, such as an immunostimulating gene, an interferon-related gene, or an apoptosis-related gene.

The term “myxoma poxvirus” refers to a myxoma poxvirus strain, which is not pathogenic to a human or a non-rabbit animal. Specific animal poxviruses affect only specific animal species (Kim M., 2015). In other words, the viruses do not cause diseases in a human or non-tropismic animal in nature. In some embodiments, myxoma poxvirus includes at least one of a wild-type squirrel poxvirus strain and a manipulated squirrel poxvirus strain containing a therapeutic gene for targeting cancer, such as an immunostimulating gene, an interferon-related gene, or an apoptosis-related gene.

Squirrel poxvirus and/or myxoma poxvirus may be manufactured using standard techniques known in the related art (Kim M., 2015). The squirrel poxvirus and/or myxoma poxvirus may be manufactured through a process that does not require a high biological safety level (BSL), and each virus has a unique progeny production cycle showing that viral genome replication occurs entirely in the cytoplasmic region of sensitive cells, thereby preventing the generation of a new hybrid virus from the host human genome during oncolysis by combined viral therapy. As a result, drug preparation and development are feasible.

In some embodiments, the squirrel poxvirus may be contained at 3×10⁵ to 10×10⁵ TCID₅₀. In some embodiments, the myxoma poxvirus may be contained at 0.1×10⁵ to 10×10⁵ TCID₅₀. In some embodiments, the potency ratio of the squirrel poxvirus to the myxoma poxvirus in the combination of the squirrel poxvirus and the myxoma poxvirus may be 1:1 to 100:1 (squirrel poxvirus:myxoma poxvirus). More preferably, the potency ratio is 1:1 to 10:1 (squirrel poxvirus:myxoma poxvirus). Even more preferably, the potency ratio is 5:1 to 10:1 (squirrel poxvirus:myxoma poxvirus).

Here, the “tissue culture infective dose so (TCID₅₀)” is a virus quantifying method, also called an endpoint dilution assay. Usually, this method is used to measure the virulence of a virus that does not form a plaque or a specific virus for an egg and an animal. Generally, this is obtained by expressing the dilution factor of viruses infecting 50% by inoculating 5 or more (usually 8 to 10 test units) cell monolayers, eggs or animals with a viral suspension diluted 10-fold by dilution as a titer. The percentage (%) of infected wells is calculated by observing the cell monolayer inoculated with the viruses by dilution to determine the presence or absence of CPE. The 50% endpoint is calculated using a Reed-Muench method or a Spearman-Karber method.

The “biological sample” refers to any biological sample, for example, adult stem cells (adipose cell-derived stem cells, or bone marrow stem cells) or cord blood stem cells, obtained from an individual, a cell line, tissue culture, or other cell sources. Methods of obtaining tissue biopsies and body fluids from mammals are well known in the art. For example, the adipose cell-derived stem cells may be pre-treated with oncolytic viruses and administered to a cancer patient.

In some embodiments, the biological sample may be prepared to kill multiple cancer cells by applying an effective amount of the combination of squirrel poxvirus and myxoma poxvirus to an ex vivo biological sample. In some embodiments, the biological sample is a bone marrow sample or a blood sample.

The term “oncolytic potential” used herein includes lysis or cell apoptosis or cell death caused by other cell death mechanisms, in addition to the prevention of cell growth or division.

The term “subject” or “patient” used herein refers to any individual member of the animal kingdom, including a human. Examples of the mammals may include, but are not limited to, any members of the class Mammalia, such as a human, non-human primates such as chimpanzee and other apes, monkey species; farm animals such as a cow, a horse, sheep, a goat and a pig; and livestock such as a rabbit, a dog and a cat. In some embodiments of the method and composition provided in the present invention, the mammals include a human or a non-human animal.

The term “treat” or “treating” used herein refers to an approach to obtain a beneficial or desirable result, including a clinical result. As a beneficial or desirable clinical result, the alleviation or improvement of one or more symptoms or conditions, reduction of the severity of a disease, stabilization of a disease condition, prevention of the development of a disease, prevention of the spread of a disease, delay or slowdown of the progression of a disease, delay or slowdown of the initiation of a disease, improvement or alleviation of a disease condition, and remission (partial or total), which may be or may not be measurable, may be included, but the present invention is not limited thereto. The “treating” may mean prolonging a patient's life to more than expected in the absence of treatment. In addition, the “treating” may mean suppressing and temporarily reducing the progression of a disease, but may also involve permanently suspending the progression of a disease. As can be understood by those of ordinary skill in the art, if treatment, while improving a specific disease condition, gives a greater adverse effect to a patient, compared to any benefits caused by the treatment, the result may not be beneficial or preferable. The present invention provides a method of treating cancer, which comprises administering a therapeutically effective amount of squirrel poxvirus and myxoma poxvirus (“combination of two viruses”) to a patient in need of cancer treatment. The combination of two viruses may be administered to a patient using a standard administration method. In some embodiments, the combination of two viruses may be administered to a disease site. Alternatively, the combination of two viruses may be systemically administered. In some embodiments, the combination of two viruses is administered intratumorally or by the use of virus-carrier cells, for example, autologous tumor or cytokine-activated autologous normal hematopoietic cells. In various embodiments, the combination of two viruses may be administered orally or parenterally, or by any standard method known to the art. Parenteral administration includes intravenous, intraperitoneal, subcutaneous, intramuscular, transepithelial, intranasal, intrapulmonary, intrathecal, rectal and topical administration. The parenteral administration may be continuous injection throughout a selected period.

When administered to a patient, the “effective amount” or “therapeutically effective amount” of a single virus or a combination of viruses is an amount required for reducing, improving, alleviating or stabilizing cancer, preventing the spread, slowdown or delaying the progression of cancer, or curing cancer at a reasonable benefit/risk ratio applicable for any medical treatment or prevention at a dose for a sufficient period of time. An effective dosage may be determined by the severity of a disease, the activity of an active agent, a patient's age, body weight, health condition and sex, the sensitivity to an active agent, an administration time, an administration route, the excretion rate of the composition of the present invention, a treatment duration, a drug used simultaneously or in combination with the composition of the disclosure, a pharmacological characteristic of the combination of viruses, virulence and titration of the combination of two viruses, and other factors known in the medical field. The pharmaceutical composition of the present invention may be administered independently or in combination with a known anticancer drug or a component known as one having anticancer activity. It is important to administer the composition at a minimum amount that does not cause a side effect but exhibits the maximum effect, in consideration of all of the factors.

In addition, one of ordinary skill in the art may determine a suitable amount of the combination of two viruses (squirrel poxvirus and/or myxoma poxvirus) for administration based on the above factors. The combination of two viruses may be administered initially at a suitable amount that can be adjusted as needed according to the clinical response of a patient. The effective amount of the combination of two viruses may be determined by the maximum amount of the combination of two viruses that can be safely administered experimentally, and the minimum amount of the combination of two viruses that produces a desired result.

When the combination of two viruses is administered systemically, to produce the same clinical effect as that through injection of the combination of two viruses into a disease site, it may be necessary to administer a significantly higher amount of the combination of two viruses. However, the suitable dosage level should be the minimum amount that can achieve the desired result. The concentration of the combination of two viruses to be administered may be different depending on the virulence of a specific strain(s) and the property of targeted cells. In some embodiments, a dose of approximately less than 10⁹ plaque-forming unit (“pfu”) is administered to a human patient. In various embodiments, a single dose of approximately 10² to 10¹² pfu, approximately 10² to 10⁹ pfu, approximately 10³ to 10⁸ pfu, or approximately 10⁴ to 10⁷ pfu may be administered.

The therapeutically effective amount of the combination of viruses may be repeatedly provided according to an effect of the initial therapeutic regimen. The administration is typically and periodically provided, and a random response is monitored. It will be noticed that a dose lower or higher than the indicated dose may be provided by those of ordinary skill in the art according to an administration schedule and a selected route.

The combination of two viruses may be administered independently or in combination with other therapeutic methods including chemotherapy, radiation therapy, immunotherapy or other antiviral therapies. For example, the combination of two viruses may be administered before or after primary tumor resection, or before, at the same time or after treatment such as radiation therapy or the administration of conventional chemotherapeutic drugs.

To aid administration, the combination of two viruses may be formulated as a component of the pharmaceutical composition. It is known that the composition may generally contain pharmaceutically acceptable concentrations of a salt, a buffer, a preservative, and various compatible excipients. For all delivery forms, the combination of two viruses may be formulated in a physiological salt solution. The pharmaceutical composition may additionally contain other therapeutic agents, for example, an anticancer agent. In some embodiments, the composition includes a chemotherapeutic agent.

The chemotherapeutic agent may be, for example, any agonist that substantially exhibits an oncolytic effect against cancer cells or neoplastic cells of a patient, and does not suppresses or reduces a lytic effect of the viral composition. For example, the chemotherapeutic agent may be anthracycline, an alkylating agent, an alkyl sulfonate, aziridine, ethylene imine, methylene amine, nitrogen mustard, nitrosourea, an antibiotic, an antimetabolite, a folate analog, a purine analog, a pyrimidine analog, an enzyme, podophyllotoxin, a platinum-containing agonist or a cytokine, but the present invention is not limited thereto. The chemotherapeutic agent may be known to be effective on a specific cell type, such as carcinogenic or neoplastic cells. In some embodiments, a chemotherapeutic agent such as thiotepa, a cisplatin-based compound, and a cyclophosphamide are effective in treatment of hematopoietic malignancies.

A ratio and identity of a pharmaceutically acceptable diluent are determined by a selected administration route, the compatibility with the combination of viruses, and the standards of pharmaceutical practices. Generally, the pharmaceutical composition may be formulated from components that do not significantly impair the biological characteristics of the combination of two viruses.

The pharmaceutical composition may be prepared by a known method for preparing a pharmaceutically acceptable composition suitable for administration to a patient so that an effective amount of the active ingredient (squirrel poxvirus and/or myxoma poxvirus) is combined with a pharmaceutically acceptable vehicle. The suitable vehicle is disclosed in, for example, Remington's Pharmaceutical Sciences (Remington's Pharmaceutical Sciences, Mack Publishing Company, Easton, Pa., USA 1985). Based on this, the composition includes the combination of two viruses contained in a buffer solution having an appropriate pH and the same osmotic pressure as a physiological fluid, along with one or more pharmaceutically acceptable vehicle or diluent.

The pharmaceutical composition may be administered to a patient in various forms according to a selected administration route, as known by those of ordinary skill in the art. The pharmaceutical composition of the present invention may be administered orally or parenterally. The pharmaceutical composition may be orally administered, for example, with an inactive diluent or anabolic carrier, enclosed in a hard or soft gelatin capsule, or compressed as a tablet. For oral administration, the combination of viruses may be incorporated with an excipient, and may be used in the form of edible tablets, buccal tablets, troche tablets, capsules, elixirs, suspensions, syrups, wafers or the like.

The solution of the combination of two viruses may be prepared in a physiologically suitable buffer. Under common conditions for storage and use, these formulations contain preservatives that may prevent microbial growth, but not inactivate the combination of viruses. A method of preparing a suitable formulation may be known by those of ordinary skill in the art. Conventional processes and components for selecting and preparing a suitable dosage form are disclosed in, for example, Remington's Pharmaceutical Sciences and in The United States Pharmacopeia: The National Formulary (USP 24 NF19) (1999).

In some embodiments, the pharmaceutical composition is directly administered to a disease site, for example, a tumor site using an injection (subcutaneously, intravenously or intramuscularly), orally, or alternatively percutaneously.

A form of the pharmaceutical composition suitable for an injectable use includes a sterilized powder for instant preparation of a sterilized aqueous solution or dispersion and a sterilized injectable solution or dispersion, and here, the term “sterilized” does not extend to the combination of viruses to be administered itself. All of the forms should be sterilized, and fluid enough to facilitate injection.

A dosage of the pharmaceutical composition used herein may depend on a specific disease to be treated, the severity of a disease, individual patient parameters including an age, a physical condition, a size and a body weight, a treatment duration, the features of combination therapy (if any), a specific administration route, and other similar factors within the knowledge and expertise of health personnel. These factors are known to those of ordinary skill in the art, and may be obtained by minimal basic experiments. In some embodiments, the pharmaceutical composition includes a therapeutically effective amount(s) of active agent(s) and a pharmaceutically acceptable excipient.

The combination of two viruses of the present invention is effective on cells obtained from cancer comprising oncolytic virus-resistant cancer. The term “cancer” or “cancer cell” used herein refers to cells showing abnormal growth, which are characterized by significant loss or inactivation of the control of cell proliferation. The term “cancer” may include metastatic cancer as well as non-metastatic cancer.

Examples of the cancer and/or cancer cells that can be treated by the present invention may be, but are not limited to, cells derived from patients having hematopoietic malignancies such as lymphoma, myeloma and leukemia, lung cancer, small cell lung cancer, breast cancer, colon cancer, pancreatic cancer, brain cancer, ovarian cancer and stomach cancer. In some embodiments, cancer cells treated by the above-described combination of viruses are derived from patients with a hematopoietic malignant tumor. The hematopoietic malignant tumor may be leukemia, lymphoma or myeloma. In some embodiments, the cancer cells are derived from patients having one of CML and MM. In some embodiments, the cancer is intractable cancer and/or the cancer cells are derived from “intractable cancer”. The “intractable cancer” used herein refers to cancer that has not responded to or has been resistant to treatment.

A biological sample including hematopoietic stem cells and cancer cells may be obtained from a patient through any standard process known to the art, including biopsy and aspiration, but the present invention is not limited thereto. After treatment with the combination of viruses of the present invention, the treated hematopoietic stem cells may be returned or administered to a patient using any known technique known in the art.

Previous studies revealed that oncolytic myxoma poxvirus preferentially infects and kills tumor suppressor dysfunctional cells (Kim M. et al., 2010). Hoping not to be restrained by theory, since the combination of viruses is considered to include p53, ATM and Rb tumor suppressor genes of dysfunctional cells and kill cancer cells including a defective interferon response, cancer cells may be selectively killed without damaging normal human or animal tissue cells.

The “pharmaceutically acceptable” excipient used herein refers to an excipient such as a carrier or diluent which is relatively non-toxic without eliminating the biological activity or properties of an active agent, that is, which may be administered to an individual without causing an undesirable biological effect or interacting with any of components of the composition in a harmful method.

The pharmaceutical composition used herein refers to the combination of viruses as an active agent(s) containing pharmaceutically acceptable other chemical components, that is, excipients such as a carrier, a stabilizer, a diluent, a disintegrating agent, a suspending agent, a thickening agent, a binder, an anti-microbial preservative, antioxidant and/or a buffer, but the present invention is not limited thereto.

The term “carrier” used herein refers to a relatively non-toxic chemical compound or agonist that allows an active agent(s) to be incorporated into cells or tissue. The term “diluent” refers to a chemical compound used to dilute an active agent(s) of interest before delivery. Since it provides a more stable environment, the diluent may be used to stabilize the active agent(s).

The buffer may be sued to maintain a desired pH of the pharmaceutical composition against the influence of external agonists and the equilibrium shift of the composition after being established.

In some embodiments, the pharmaceutical composition of the present invention is an injectable form. For parenteral injection, a suitable formulation may be, for example, a sterilized aqueous or non-aqueous solution containing a physiologically acceptable carrier.

In some embodiments, the pharmaceutical composition of the present invention is an oral form. For oral administration, the above-described active agent(s) may be easily formulated by combining with a pharmaceutically acceptable carrier or excipient well known in the art. The carrier allows the active agent described herein to be formulated as a tablet, a powder, a pill, a sugar-coated pill, a capsule, a liquid, a gel, a syrup, an elixir, a slurry or a suspension for oral intake by a patient to be treated.

The term “unit dosage form” refers to a physically distinct unit appropriate as a single dose, which contains a predetermined amount of an active component bound with a suitable pharmaceutical excipient, and one or more dosage forms are used throughout an entire dose therapy for generating a desired therapeutic effect of treating cancer.

The pharmaceutical composition described herein may be formed in unit doses, which are suitable for an exact dose of single administration. In the unit dosage form, the formulation is divided into unit doses containing a suitable amount of one or more active agents. The unit dose may refer to packaging containing an individual amount of each formulation. Non-limiting examples include a tablet or capsule packaged in vials or ampules, and a powder. The aqueous suspension composition may be packaged in a single-dose non-reusable container. Alternatively, a multi-dose reusable container may be used, and in this case, a preservative is typically contained in the composition. As an example, a formulation for parenteral injection may be produced as a unit dosage form including an ampoule, or in a multi-dose container, but the present invention is not limited thereto.

The above-mentioned ranges are merely suggestive because there are a larger number of variables associated with individual therapeutic regimens, and it is common to deviate significantly from these recommended values. Such doses depend on many variables, but are not limited thereto, and may vary according to the activity of an active agent(s) used, a disease or illness to be treated, an administration mode, requirements for each patient, the severity of the disease or illness to be treated, and a physician's judgement.

The present invention also relates to a sterilized glass or polyolefin container containing the pharmaceutical composition described herein. In some embodiments, the container is non-DEHP (bis(2-ethylhexyl)phthalate (di-2-ethylhexyl phthalate, diethylhexyl phthalate), DEHP; dioctyl phthalate, DOP) or non-polyvinylpyrrolidone (PVP).

The kit includes a suitable container, for example, a box, an individual bottle, bag or ampoule. The suspension composition may be packaged in a single-dose non-reusable container or a multi-dose reusable container.

The term “co-administration” used herein refers to administration of two viruses (active agents) to a single patient, and is intended to encompass a therapeutic regimen in which agonists are administered by the same or different routes or at the same or different times.

The composition containing the active agent(s) described herein may be administered for preventive and/or therapeutic treatment. In a therapeutic application, the composition is administered to a patient already having a disease or illness at an amount sufficient to treat or at least partially arrest symptoms of the disease or illness. The effective amount for this use will be dependent on the severity and progression of a disease or illness, a conventionally-used therapeutic method, a patient's health condition, body weight and response to the active agents, and a physician's judgement. It is considered that such a therapeutically effective amount is determined by the proficiency of one of ordinary skill in the art by basic experimentation (including but not limited to a dose-increasing clinical trial).

In a preventive application, the composition containing the active agent(s) described herein is administered to a patient prone to cancer or otherwise having a risk of cancer. A dose of the composition is defined as a “prophylactically effective amount or dose”. In this use, an exact amount also depends on a patient's health condition or body weight. It is considered that the prophylactically effective amount is determined by the proficiency of one of ordinary skill in the art by basic experimentation (for example, dose-increasing clinical trial). When used in a patient, the effective amount for this use may be dependent on the severity and process of a disease, disorder or illness, a conventional therapeutic method, a patient's health condition and response to the active agents, and a physician' judgement.

When the patient's condition is not improved, as the physician's discretion, the administration of the above-described active agents may be performed chronically, that is, for an extended period of time including the entire lifetime of the patient in order to ameliorate or otherwise control or limit the symptoms of the patient's disease or illness.

An amount of the given agonists corresponding to the effective amount may vary according to parameters such as a specific active agent, the stage and severity of a disease, the identity of a subject or host in need of treatment (e.g., an age, a body weight, a sex, etc.). Nevertheless, it can be basically determined by a method known in the art according to specific circumstances surrounding a case, including, for example, a specific active agent to be administered, an administration route, a disease to be treated, and a subject or host to be treated.

### EXAMPLES

### Example 1: Oncolytic Effect of Squirrel Poxvirus in Gynecological Carcinoma

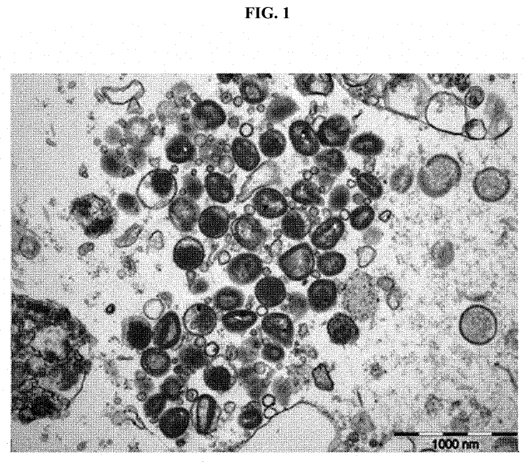

As shown in FIGS. 1, 2 to 4 days after wild-type squirrel poxvirus infection (10 MOI), upon squirrel poxvirus challenge, the virus was detected in the cytoplasmic area of C33A human cervical cancer cells. Mature/immature squirrel poxvirus particles were observed under a high-power electron microscope (EM; EM magnification: 30,000×).

### Example 2: Oncolytic Effect of Squirrel Poxvirus in Hepatocellular Carcinoma

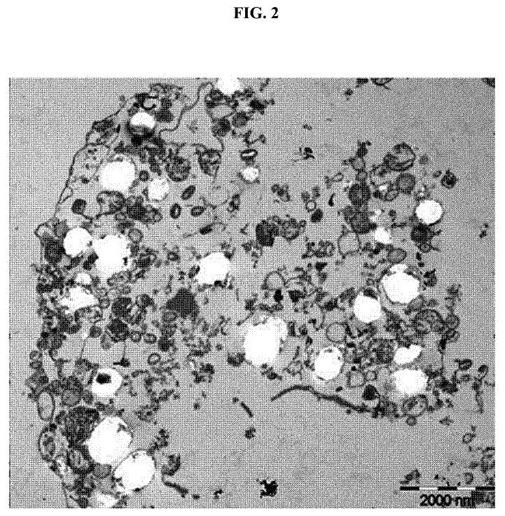

As shown in FIG. 2, 2 to 4 days after the infection of wild-type squirrel poxvirus (10 MOI), upon squirrel poxvirus challenge, the virus was detected in the cytoplasmic area of Hep3B human hepatocellular carcinoma cells. Mature/immature squirrel poxvirus particles were observed under a high-power electron microscope (EM; EM magnification: 60,000×).

### Example 3: Oncolytic Effect of Squirrel Poxvirus in Stomach Cancer

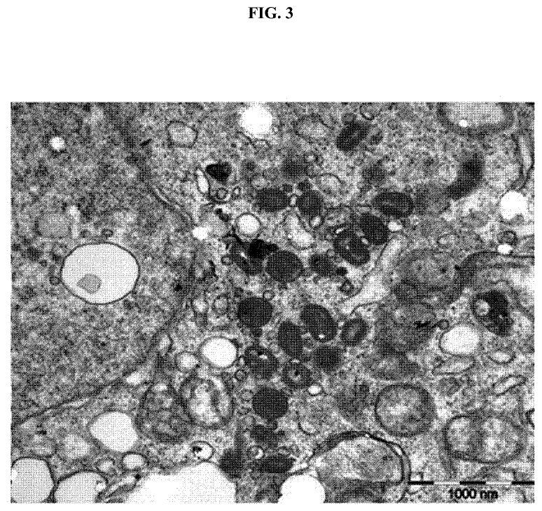

As shown in FIG. 3, 2 to 4 days after the infection of wild-type squirrel poxvirus (10 MOI), upon squirrel poxvirus challenge, the virus was detected in the cytoplasmic area of AGS3 human stomach cancer cells. Mature/immature squirrel poxvirus particles were observed under a high-power electron microscope (EM; EM magnification: 30,000×).

### Example 4: Synergistic Oncolytic Effect of Squirrel Poxvirus and Myxoma Poxvirus in Hepatocellular Carcinoma

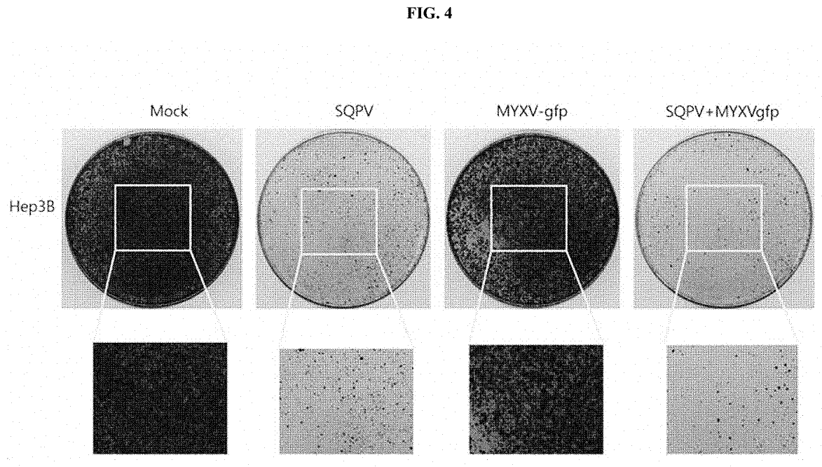

Hep3B cells were infected with wild-type squirrel poxvirus (8×10⁵ TCID₅₀) or myxoma poxvirus (1×10⁵ TCID₅₀). Six days after viral infection, cell colonies were immobilized, and stained with a crystal violet solution. As shown in FIG. 4, the colonies were photographed using a high-power scanner. The Hep3B hepatic cells were killed by SQPV injection and MYXV-gfp infection. In the pictures, as the number of dots decreases, more cancer cells were killed. It was confirmed that the number of dots was considerably reduced by co-infection of SQPV and MYXV-gfp (SQPV: squirrel poxvirus; MYXVgfp: gfp-expressing myxoma poxvirus).

### Example 5: Synergistic Oncolytic Effect of Squirrel Poxvirus and Myxoma Poxvirus in Lung Cancer

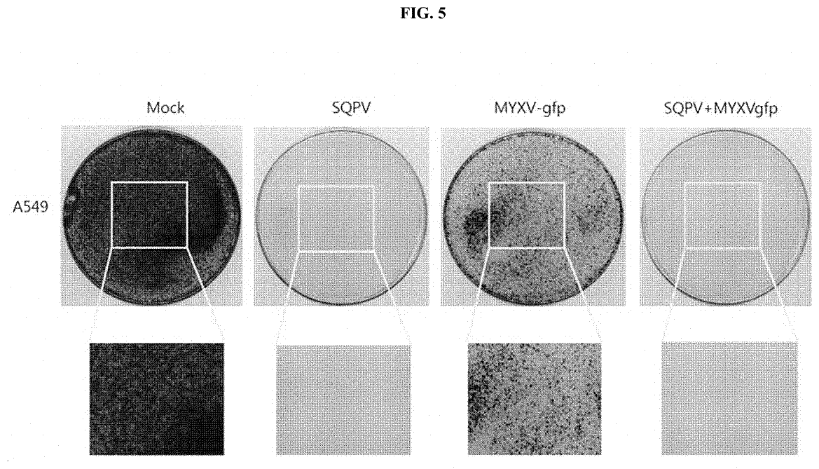

A549 cells were infected with wild-type squirrel poxvirus (8×10⁵ TCID₅₀) or myxoma poxvirus (1×10⁵ TCID₅₀). Six days after viral infection, cell colonies were immobilized, and stained with a crystal violet solution. As shown in FIG. 5, the colonies were photographed using a high-power scanner. The A549 lung cells were killed by SQPV injection and MYXV-gfp infection. In the pictures, as the number of dots decreases, more cancer cells were killed. It was confirmed that the number of dots was considerably reduced by co-infection of SQPV and MYXV-gfp (SQPV: squirrel poxvirus; MYXVgfp: gfp-expressing myxoma poxvirus).

### Example 6: Synergistic Oncolytic Effect of Squirrel Poxvirus and Myxoma Poxvirus in Breast Cancer Cells

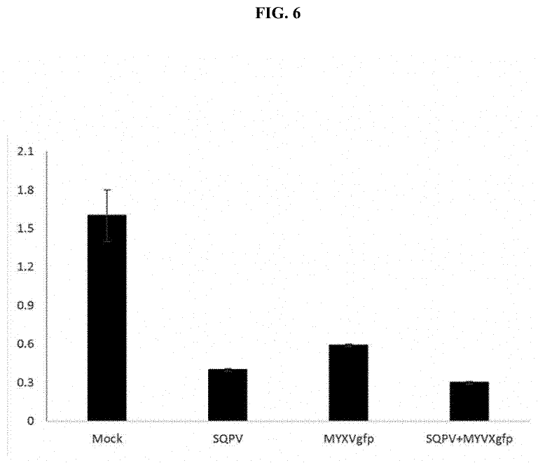

MCF7 cells were infected with either wild-type squirrel poxvirus (8×10⁵ TCID₅₀) or myxoma poxvirus (1×10⁵ TCID₅₀), or a combination of the two viruses (SQPV: 4×10⁵ TCID₅₀, MYXVgfp: 0.5×10⁵ TCID₅₀). Six days after viral infection, as shown in FIG. 6, a cell survival rate was evaluated using a WST-1 assay according to the manufacturer's instructions. The MCF7 breast cancer cells were killed by SQPV infection and MYXV-gfp infection. As a result, compared with treatment with single viruses, it was confirmed that the treatment with the combination of two type of viruses exhibits a synergistic oncolytic effect (SQPV: squirrel poxvirus, MYXVgfp: gfp-expressing myxoma poxvirus).

TABLE 1
MCF7 MTT assay

| | | | | Avg | SD |
|---|---|---|---|---|---|
| Mock | 1.6 | 1.4 | 1.8 | 1.6 | 0.2 |
| SQPV | 0.4 | 0.39 | 0.41 | 0.4 | 0.01 |
| MYXVgfp | 0.59 | 0.58 | 0.6 | 0.59 | 0.01 |
| SQPV + MYVXgfp | 0.3 | 0.29 | 0.31 | 0.3 | 0.01 |

### Example 7: Synergistic Oncolytic Effect of Squirrel Poxvirus and Myxoma Poxvirus in Squirrel Poxvirus-Resistant Cancer

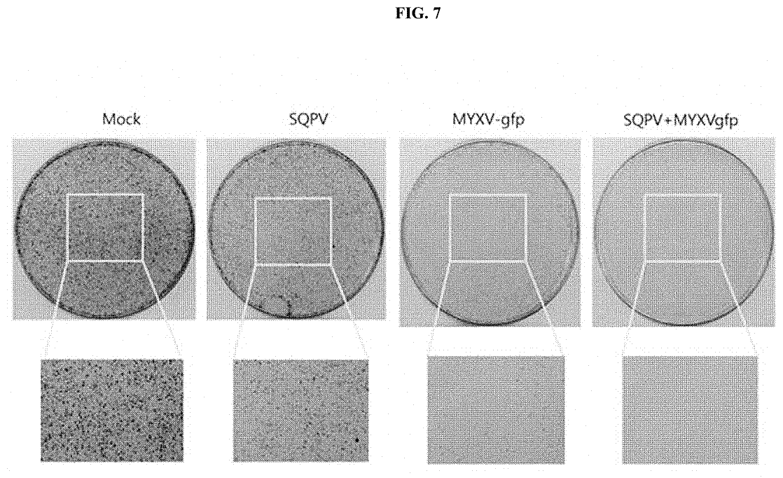

Squirrel poxvirus-resistant HTR1 cells (Kim et al., 2007) were infected with either wild-type squirrel poxvirus (8×10⁵ TCID₅₀) or myxoma poxvirus (1×10⁵ TCID₅₀) or a combination of two viruses (SQPV: 4×10⁵TCID₅₀, MYXV or MYXVgfp: 0.5×10⁵ TCID₅₀). Six days after viral infection, cell colonies were immobilized and stained with a crystal violet solution. As shown in FIG. 7, the colonies were photographed using a high-power scanner. In the pictures, as the number of dots decreases, more cancer cells were killed. The combination of the SQPV and MYXV-gfp infection synergistically increased the death of HTR1 cancer cells (SQPV: wild-type squirrel poxvirus, MYXVgfp: gfp-expressing myxoma poxvirus).

### Example 8: Synergistic Oncolytic Effect of Squirrel Poxvirus and Myxoma Poxvirus in In Vivo B16F10-Enhanced Model

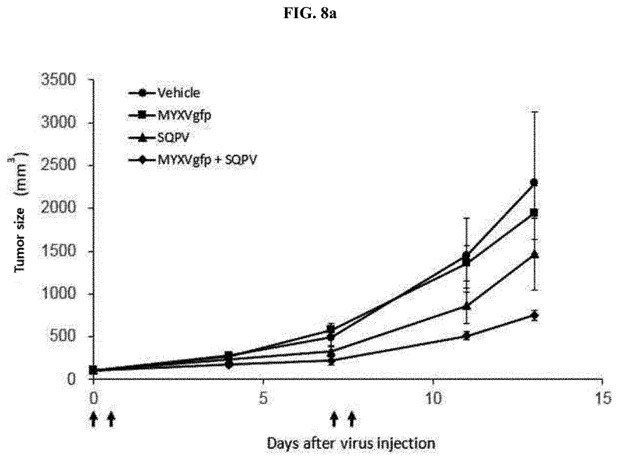

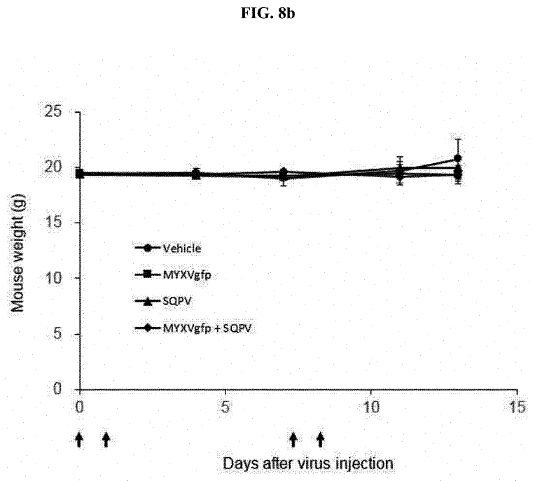

B16F10 murine melanoma cells (1×10⁵ cells per mouse) were subcutaneously injected into right flank of age-matched (5-week-old) female C57BL/6 mice. When the average of tumor volumes reached approximately 100 to 120 mm³, the mice were randomly assigned to a treatment group. Viruses (squirrel poxvirus and myxoma poxvirus) or a PBS vehicle were injected into tumors on day 1, 2, 8 and 9. A tumor volume (FIG. 8A) and a mouse weight (FIG. 8B) were measured at regular intervals until tumors grew to approximately 2,000 mm³. The combination treatment of squirrel poxvirus and myxoma poxvirus synergistically induced tumor growth suppression in B16F10 melanoma tumors (lowest line), compared with single squirrel poxvirus infection (filled triangle, third line from top) and single myxoma poxvirus infection (filled rectangle, second line from top). As shown in FIG. 8B, as any sign and symptom of systemic toxicity were not observed in mice treated with the combination of squirrel poxvirus and myxoma poxvirus, compared with those of the vehicle-treated mice, it was confirmed that either single treatment or combination treatment is not toxic to mice infected with the virus (SQPV: squirrel poxvirus, MYXV gfp: gfp-expressing myxoma poxvirus).

The disclosures in the references cited throughout the specification are incorporated herein by reference in their entirety without being inconsistent with the disclosures of the present invention. It should be appreciated that the examples and embodiments of the present invention are merely provided to exemplify the present invention, and in this regard, it should be understood that various modifications or alterations will be suggested by those of ordinary skill in the art, and included in the spirit and scope of the present application.

The above description of specific embodiments, by applying knowledge within the proficiency in the art, may entirely reveal the general property of the present invention in which these specific embodiments can be easily modified and/or adjusted for various applications without unnecessary experiments conducted by other people and without departing from the general concept of the present invention. Therefore, such adjustments and modifications are intended to be in the definitions and ranges of equivalents of the disclosed embodiments, based on the disclosures and guidance provided herein. It should be understood that the expression or terminology used herein is for describing the present invention, not for limiting, so that the terminology or expression used herein may be interpreted by those of ordinary skill in the art in aspects of the disclosures and guidance.

The range and scope of the present invention should not be limited by any of the exemplary embodiments herein, and should be defined by the claims and equivalents thereof.

All of the various aspects, embodiments and options disclosed herein may be combined in any and all variations.

All publications, patents and patent applications mentioned herein are incorporated herein by reference to the same extent as being represented that each publication, patent or patent application is specifically and individually incorporated by reference.

1. A method of treating cancer, which comprises administering a therapeutically effective amount of (a) a combination of squirrel poxvirus and myxoma poxvirus or (b) a biological sample treated with (a) to a patient in need thereof.

2. The method of claim 1, wherein the squirrel poxvirus is wild-type squirrel poxvirus.

3. The method of claim 1, wherein the squirrel poxvirus is a therapeutic gene-expressing squirrel poxvirus for targeting cancer.

4. The method of claim 1, wherein the myxoma poxvirus is wild-type myxoma poxvirus.

5. The method of claim 1, wherein the myxoma poxvirus is a therapeutic gene-expressing myxoma poxvirus for targeting cancer.

6. The method of claim 1, wherein the squirrel poxvirus is contained at 3×10⁵ to 10×10⁵ TCID₅₀.

7. The method of claim 1, wherein the myxoma poxvirus is contained at 0.1×10⁵ to 10×10⁵ TCID₅₀.

8. The method of claim 1, wherein a potency ratio of the squirrel poxvirus to the myxoma poxvirus in the combination of the squirrel poxvirus and the myxoma poxvirus is 1:1 to 100:1 (squirrel poxvirus:myxoma poxvirus).

9. The method of claim 1, wherein the biological sample is prepared to kill a plurality of cancer cells by applying an effective amount of the composition of squirrel poxvirus and myxoma poxvirus to an ex vivo biological sample.

10. The method of claim 1, wherein the biological sample is a bone marrow or blood sample.

11. The method of claim 1, wherein the cancer is cancer having a tumor suppressor-deficient function.

12. The method of claim 1, wherein the cancer is selected from hematopoietic malignancies, lung cancer, liver cancer, breast cancer, colon cancer, pancreatic cancer, brain cancer, gynecological cancer or stomach cancer.

13. The method of claim 1, wherein the cancer is a hematopoietic malignancy selected from the group consisting of myeloproliferative disorders such as lymphoma, myeloma, myelodysplastic syndrome (MDS) and polycythemia vera, and leukemia.

14. The method of claim 1, wherein the cancer comprises malignant cells and/or benign cells.

15. The method of claim 1, wherein the cancer is intractable cancer comprising oncolytic virus-resistant cancer.

16. The method of claim 1, wherein the administering is an injectable form.

17. The the method of claim 1, wherein the administering is an oral form.

18.-19. (canceled)
